(19) United States
(12) Patent Application Publication
(10) Pub. No.: US 2016/0331442 A1
(43) Pub. Date: Nov. 17, 2016

(54) BIPOLAR FORCEPS WITH ACTIVE COOLING

(71) Applicant: Kogent Surgical, LLC, Chesterfield, MO (US)

(72) Inventors: Gregg D. Scheller, Wildwood, MO (US); Brett D. Smith, St. Louis, MO (US); Anthony E. Bramblett, Clayton, MO (US)

(21) Appl. No.: 15/092,011

(22) Filed: Apr. 6, 2016

Related U.S. Application Data
(60) Provisional application No. 62/161,059, filed on May 13, 2015.

(51) Int. Cl. A61B 18/14

(52) U.S. Cl. CPC A61B 18/1442; A61B 2018/00023

(57) ABSTRACT

A bipolar forceps with active cooling may include a first forceps arm having a first forceps arm distal end and a first forceps arm proximal end, a second forceps arm having a second forceps arm distal end and a second forceps arm proximal end, a coolant multiplexer, a coolant transfer tube, and a coolant transfer machine interface. The coolant transfer machine interface may be configured to interface with a coolant transfer machine to circulate a coolant through an internal conduit of the first forceps arm and an internal conduit of the second forceps arm. Circulating the coolant through the internal conduit of the first forceps arm may be configured to decrease a temperature of a conductor tip of the first forceps arm. Circulating the coolant through the internal conduit of the second forceps arm may be configured to decrease a temperature of a conductor tip of the second forceps arm.

### CROSS-REFERENCE TO RELATED APPLICATIONS

This application claims the benefit of U.S. Provisional Application No. 62/161,059, filed May 13, 2015.

### FIELD OF THE INVENTION

The present disclosure relates to a medical device, and, more particularly, to an electrosurgical instrument.

### BACKGROUND OF THE INVENTION

A variety of complete surgical procedures and portions of surgical procedures may be performed with bipolar forceps, e.g., bipolar forceps are commonly used in dermatological, gynecological, cardiac, plastic, ocular, spinal, maxillofacial, orthopedic, urological, and general surgical procedures. Bipolar forceps are also used in neurosurgical procedures; however, the use of bipolar forceps in neurosurgical procedures presents unique risks to patients if the surgeon is unable to both visually and tactilely confirm that an electrosurgical procedure is being performed as intended. Accordingly, there is a need for a bipolar forceps that allows a surgeon to both visually and tactilely confirm that an electrosurgical procedure is being performed as intended. After an electrosurgical procedure is performed as intended, cauterized tissue may adhere to the electrodes of the bipolar forceps which must be removed before another electrosurgical procedure may be perm formed effectively. Accordingly, there is a need for a bipolar forceps that reduces adherence of cauterized tissue to electrodes.

### BRIEF SUMMARY OF THE INVENTION

The present disclosure presents a bipolar forceps with active cooling. In one or more embodiments, a bipolar forceps with active cooling may comprise a first forceps arm having a first forceps arm distal end and a first forceps arm proximal end, a second forceps arm having a second forceps arm distal end and a second forceps arm proximal end, a coolant multiplexer, a coolant transfer tube, and a coolant transfer machine interface. Illustratively, the coolant transfer machine interface may be configured to interface with a coolant transfer machine to circulate a coolant through an internal conduit of the first forceps arm and an internal conduit of the second forceps arm. In one or more embodiments, circulating the coolant through the internal conduit of the first forceps arm may be configured to decrease a temperature of a conductor tip of the first forceps arm. In one or more embodiments, circulating the coolant through the internal conduit of the second forceps arm may be configured to decrease a temperature of a conductor tip of the second forceps arm.

### BRIEF DESCRIPTION OF THE DRAWINGS

The above and further advantages of the present invention may be better understood by referring to the following description in conjunction with the accompanying drawings in which like reference numerals indicate identical or functionally similar elements:

FIGS. 1A, 1B, 1C, 1D, and 1E are schematic diagrams illustrating a forceps arm;

FIG. 2 is a schematic diagram illustrating an exploded view of a bipolar forceps with active cooling assembly;

FIG. 3 is a schematic diagram illustrating an assembled bipolar forceps with active cooling;

FIGS. 4A, 4B, and 4C are schematic diagrams illustrating a gradual closing of a bipolar forceps with active cooling;

FIGS. 5A, 5B, and 5C are schematic diagrams illustrating a uniform compression of a vessel.

### DETAILED DESCRIPTION OF AN ILLUSTRATIVE EMBODIMENT

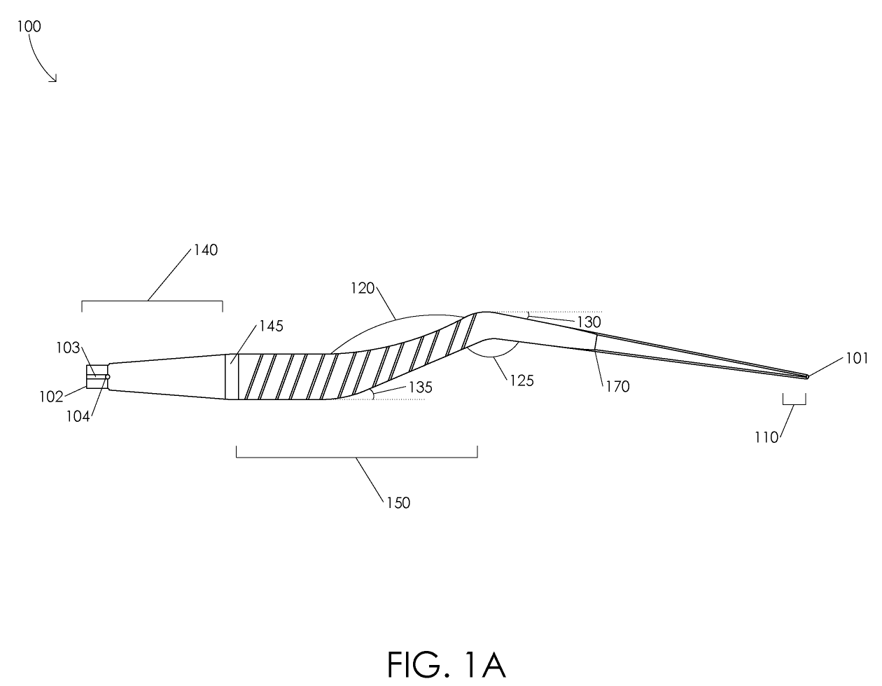

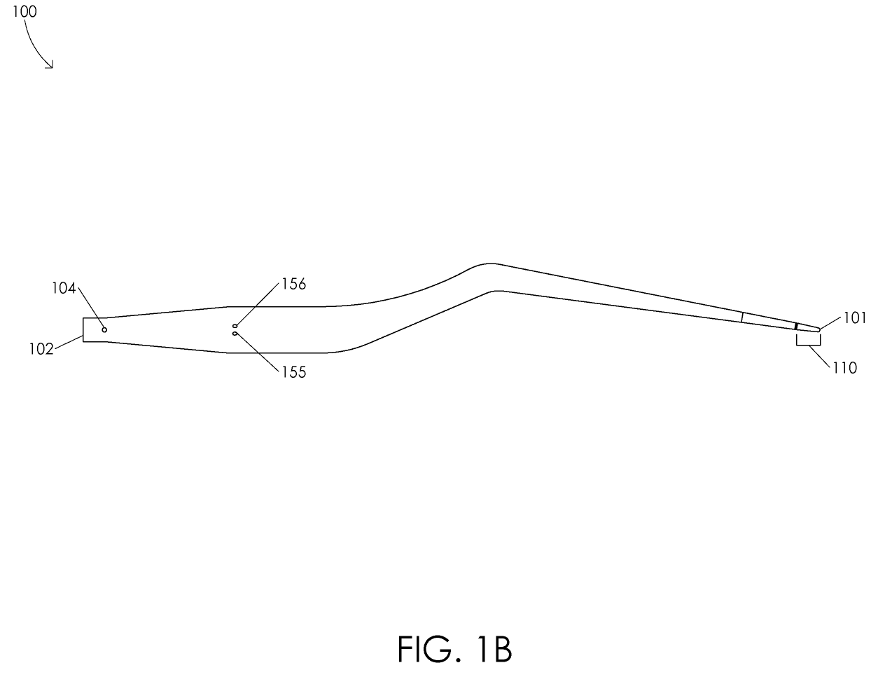

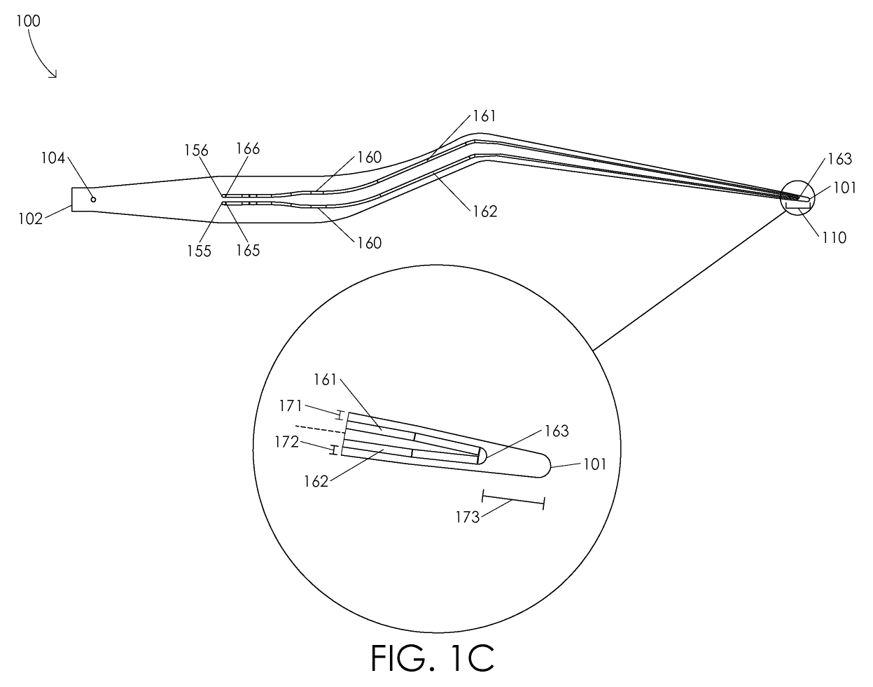

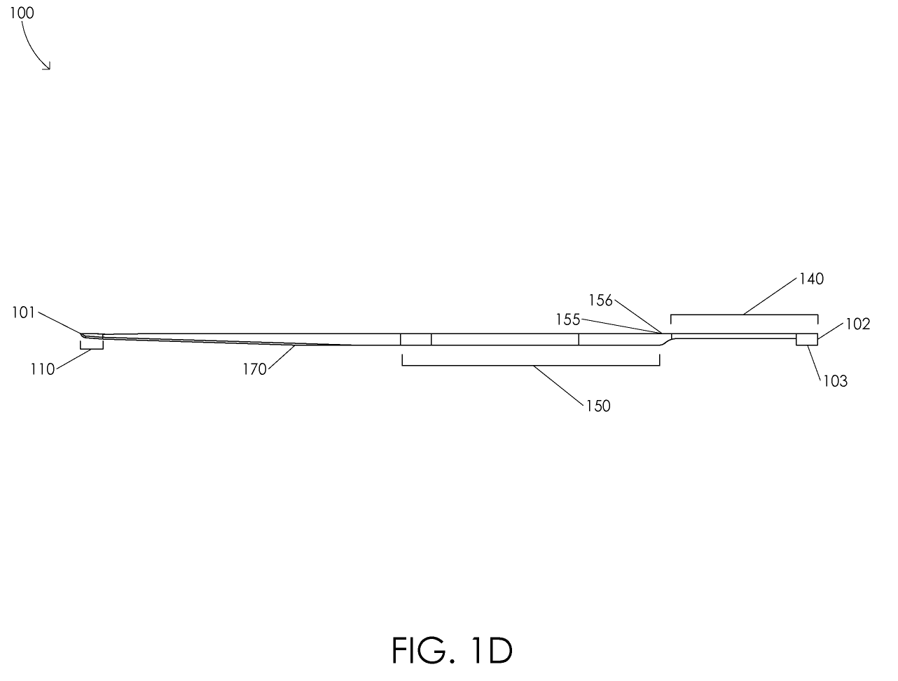

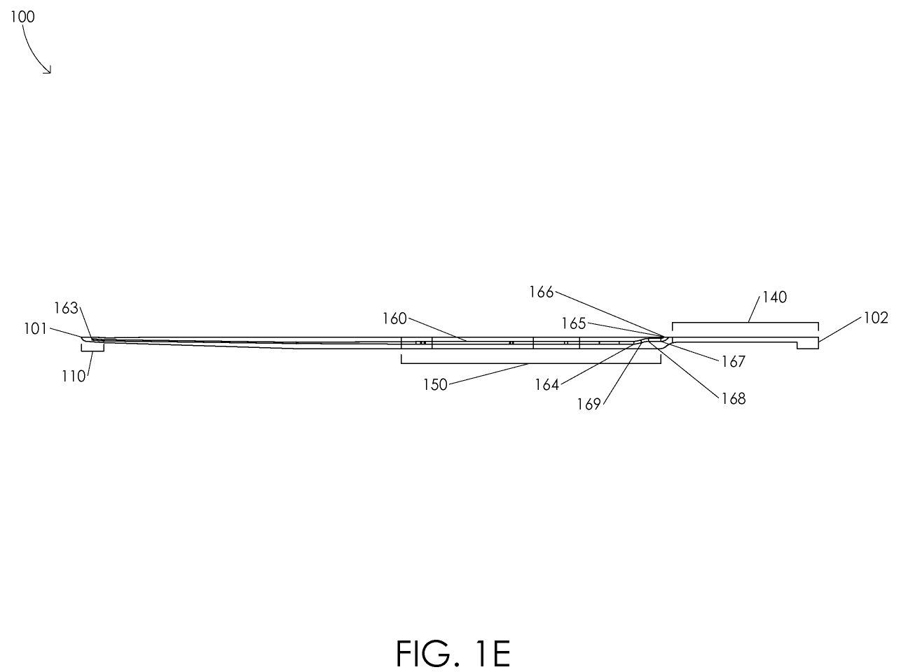

FIGS. 1A, 1B, 1C, 1D, and 1E are schematic diagrams illustrating a forceps arm 100. FIG. 1A is a schematic diagram illustrating a lateral view of a forceps arm 100. Illustratively, a forceps arm 100 may comprise a forceps arm distal end 101, a forceps arm proximal end 102, an input conductor housing 103, an input conductor interface 104, a conductor tip 110, a forceps arm superior incline angle 120, a forceps arm inferior decline angle 125, a forceps arm superior decline angle 130, a forceps arm inferior incline angle 135, a socket interface 140, an internal conduit opening platform 145, a forceps arm grip 150, and a forceps jaw taper interface 170. FIG. 1B is a schematic diagram illustrating a medial view of a forceps arm 100. Illustratively, a forceps arm 100 may comprise an inferior opening 155 and a superior opening 156. In one or more embodiments, forceps arm 100 may be may be manufactured from any suitable material, e.g., polymers, metals, metal alloys, etc., or from any combination of suitable materials. Illustratively, forceps arm 100 may be manufactured from an electrically conductive material, e.g., metal, graphite, conductive polymers, etc. In one or more embodiments, forceps arm 100 may be manufactured from an electrically conductive metal, e.g., silver, copper, gold, aluminum, etc. Illustratively, forceps arm 100 may be manufactured from an electrically conductive metal alloy, e.g., a silver alloy, a copper alloy, a gold alloy, an aluminum alloy, stainless steel, etc.

In one or more embodiments, forceps arm 100 may be manufactured from a material having an electrical conductivity in a range of 30.0×10⁶ to 40.0×10⁶ Siemens per meter at a temperature of 20.0° C., e.g., forceps arm 100 may be manufactured from a material having an electrical conductivity of 35.5×10⁶ Siemens per meter at a temperature of 20.0° C. Illustratively, forceps arm 100 may be manufactured from a material having an electrical conductivity of less than 30.0×10⁶ Siemens per meter or greater than 40.0×10⁶ Siemens per meter at a temperature of 20.0° C. In one or more embodiments, forceps arm 100 may be manufactured from a material having a thermal conductivity in a range of 180.0 to 250.0 Watts per meter Kelvin at a temperature of 20.0° C., e.g., forceps arm 100 may be manufactured from a material having a thermal conductivity of 204.0 Watts per meter Kelvin at a temperature of 20.0° C. Illustratively, forceps arm 100 may be manufactured from a material having a thermal conductivity of less than 180.0 Watts per meter Kelvin or greater than 250.0 Watts per meter Kelvin at a temperature of 20.0° C. In one or more embodiments, forceps arm 100 may be manufactured from a material having an electrical conductivity in a range of 30.0×10⁶ to 40.0×10⁶ Siemens per meter and a thermal conductivity in a range of 180.0 to 250.0 Watts per meter Kelvin at a temperature of 20.0° C., e.g., forceps arm 100 may be manufactured from a material having an electrical conductivity of 35.5×10⁶ Siemens per meter and a thermal conductivity of 204.0 Watts per meter Kelvin at a temperature of 20.0° C.

Illustratively, forceps arm 100 may have a density in a range of 0.025 to 0.045 pounds per cubic inch, e.g., forceps arm 100 may have a density of 0.036 pounds per cubic inch. In one or more embodiments, forceps arm 100 may have a density less than 0.025 pounds per cubic inch or greater than 0.045 pounds per cubic inch. For example, forceps arm 100 may have a density of 0.0975 pounds per cubic inch. Illustratively, forceps arm 100 may have a mass in a range of 0.0070 to 0.0092 pounds, e.g., forceps arm 100 may have a mass of 0.0082 pounds. In one or more embodiments, forceps arm 100 may have a mass less than 0.0070 pounds or greater than 0.0092 pounds. Illustratively, forceps arm 100 may have a volume in a range of 0.20 to 0.26 cubic inches, e.g., forceps arm 100 may have a volume of 0.227 cubic inches. In one or more embodiments, forceps arm 100 may have a volume less than 0.20 cubic inches or greater than 0.26 cubic inches. Illustratively, forceps arm 100 may have a surface area in a range of 5.0 to 8.0 square inches, e.g., forceps arm 100 may have a surface area of 6.9 square inches. In one or more embodiments, forceps arm 100 may have a surface area less than 5.0 square inches or greater than 8.0 square inches. Illustratively, conductor tip 110 may have a surface area in a range of 0.03 to 0.07 square inches, e.g., conductor tip 110 may have a surface area of 0.053 square inches. In one or more embodiments, conductor tip 110 may have a surface area less than 0.03 square inches or greater than 0.07 square inches. Illustratively, a ratio of forceps arm 100 surface area to conductor tip 110 surface area may be in a range of 100.0 to 180.0, e.g., a ratio of forceps arm 100 surface area to conductor tip 110 surface area may be 137.9. In one or more embodiments, a ratio of forceps arm 100 surface area to conductor tip 110 surface area may be less than 100.0 or greater than 180.0.

Illustratively, conductor tip 110 may be configured to prevent tissue from sticking to conductor tip 110. In one or more embodiments, conductor tip 110 may comprise an evenly polished material configured to prevent tissue sticking. Illustratively, conductor tip 110 may be polished and then subjected to a surface treatment process configured to prevent tissue sticking, e.g., conductor tip 110 may be coated by a material configured to prevent tissue sticking. In one or more embodiments, conductor tip 110 may be subjected to a chemical surface treatment process configured to prevent tissue sticking. Illustratively, conductor tip 110 may be subjected to a plasma surface treatment process configured to prevent tissue sticking. In one or more embodiments, conductor tip 110 may be subjected to a particle deposition surface treatment process configured to prevent tissue sticking. Illustratively, conductor tip 110 may be subjected to a vapor deposition surface treatment process configured to prevent tissue sticking. In one or more embodiments, conductor tip 110 may be subjected to a surface treatment process configured to increase a contact angle between water and a surface of conductor tip 110, e.g., conductor tip 110 may be subjected to a surface treatment process configured to increase a hydrophobicity of a surface of conductor tip 110 to prevent tissue sticking. Illustratively, conductor tip 110 may be modified wherein a contact angle between a water droplet and a surface of conductor tip 110 is in a range of 130.0 to 175.0 degrees, e.g., conductor tip 110 may be modified wherein a contact angle between a water droplet and a surface of conductor tip 110 is 165.0 degrees. In one or more embodiments, conductor tip 110 may be modified wherein a contact angle between a water droplet and a surface of conductor tip 110 is less than 130.0 degrees or greater than 175.0 degrees. Illustratively, conductor tip 110 may be subjected to a surface treatment process configured to decrease a contact angle between water and a surface of conductor tip 110, e.g., conductor tip 110 may be subjected to a surface treatment process configured to increase a hydrophilicity of a surface of conductor tip 110 to prevent tissue sticking. In one or more embodiments, conductor tip 110 may be modified wherein a contact angle between a water droplet and a surface of conductor tip 110 is in a range of 5.0 to 40.0 degrees, e.g., conductor tip 110 may be modified wherein a contact angle between a water droplet and a surface of conductor tip 110 is 25.0 degrees. Illustratively, conductor tip 110 may be modified wherein a contact angle between a water droplet and a surface of conductor tip 110 is less than 5.0 degrees or greater than 40.0 degrees.

In one or more embodiments, a surface of conductor tip 110 may have a roughness average in a range of 25.0 to 150.0 nanometers, e.g., a surface of conductor tip 110 may have a roughness average of 98.8 nanometers. Illustratively, a surface of conductor tip 110 may have a roughness average of less than 25.0 nanometers or greater than 150.0 nanometers. In one or more embodiments, a surface of conductor tip 110 may have a root mean square average between height deviations over a total surface area of conductor tip 110 in a range of 30.0 to 150.0 nanometers, e.g., a surface of conductor tip 110 may have a root mean square average between height deviations over a total surface area of conductor tip 110 of 112.0 nanometers. Illustratively, a surface of conductor tip 110 may have a root mean square average between height deviations over a total surface area of conductor tip 110 of less than 30.0 nanometers or greater than 150.0 nanometers. In one or more embodiments, a surface of conductor tip 110 may have an average maximum profile of the ten greatest peak-to-valley separations over a total surface area of conductor tip 110 in a range of 100.0 to 850.0 nanometers, e.g., a surface of conductor tip 110 may have an average maximum profile of the ten greatest peak-to-valley separations over a total surface area of conductor tip 110 of 435.0 nanometers. Illustratively, a surface of conductor tip 110 may have an average maximum profile of the ten greatest peak-to-valley separations over a total surface area of conductor tip 110 of less than 100.0 nanometers or greater than 850.0 nanometers. In one or more embodiments, a surface of conductor tip 110 may have a maximum height difference between a highest point and a lowest point of a total surface area of conductor tip 110 in a range of 200.0 to 1300.0 nanometers, e.g., a surface of conductor tip 110 may have a maximum height difference between a highest point and a lowest point of a total surface area of conductor tip 110 of 650.0 nanometers. Illustratively, a surface of conductor tip 110 may have a maximum height difference between a highest point and a lowest point of a total surface area of conductor tip 110 of less than 200.0 nanometers or greater than 1300.0 nanometers.

In one or more embodiments, conductor tip 110 may be immersed in a chemical configured to produce a chrome conversion coating on conductor tip 110 to prevent tissue from sticking to conductor tip 110 during a surgical procedure. For example, conductor tip 110 may comprise a chromate conversion coating configured to prevent tissue from sticking to conductor tip 110 during a surgical procedure. Illustratively, conductor tip 110 may be immersed in a phosphoric acid based cleaner and then immersed in a chromic acid based coating chemical to produce a chrome conversion coating on conductor tip 110. In one or more embodiments, conductor tip 110 may be polished to a mirror finish, and then immersed in a phosphoric acid based cleaner, and then immersed in a chromic acid based coating chemical to produce a chrome conversion coating on conductor tip 110. Illustratively, conductor tip 110 may be immersed in, e.g., Iridite, Alodine, etc., to produce a chrome conversion coating on conductor tip 110 configured to prevent tissue from sticking to conductor tip 110 during a surgical procedure. In one or more embodiments, conductor tip 110 may comprise a chrome conversion coating configured to increase an electrical conductivity of conductor tip 110. Illustratively, conductor tip 110 may comprise a chrome conversion coating configured to reduce thermal spread to non-target tissue during a surgical procedure.

Illustratively, conductor tip 110 may have a length in a range of 0.22 to 0.3 inches, e.g., conductor tip 110 may have a length of 0.26 inches. In one or more embodiments, conductor tip 110 may have a length less than 0.22 inches or greater than 0.3 inches. Illustratively, conductor tip 110 may have a width in a range of 0.018 to 0.062 inches, e.g., conductor tip 110 may have a width of 0.04 inches. In one or more embodiments, conductor tip 110 may have a width less than 0.018 inches or greater than 0.062 inches. Illustratively, a geometry of forceps arm 100 may comprise a tapered portion, e.g., a tapered portion from forceps jaw taper interface 170 to forceps arm distal end 101. In one or more embodiments, forceps arm 100 may comprise a tapered portion having a tapered angle in a range of 3.0 to 4.5 degrees, e.g., forceps arm 100 may comprise a tapered portion having a tapered angle of 3.72 degrees. Illustratively, forceps arm 100 may comprise a tapered portion having a tapered angle of less than 3.0 degrees or greater than 4.5 degrees.

Illustratively, forceps arm 100 may comprise a material having a modulus of elasticity in a range of 9.0×10⁶ to 11.0×10⁶ pounds per square inch, e.g., forceps arm 100 may comprise a material having a modulus of elasticity of 10.0×10⁶ pounds per square inch. In one or more embodiments, forceps arm 100 may comprise a material having a modulus of elasticity less than 9.0×10⁶ pounds per square inch or greater than 11.0×10⁶ pounds per square inch. Illustratively, forceps arm 100 may comprise a material having a shear modulus in a range of 3.5×10⁶ to 4.5×10⁶ pounds per square inch, e.g., forceps arm 100 may comprise a material having a shear modulus of 3.77×10⁶ pounds per square inch. In one or more embodiments, forceps arm 100 may comprise a material having a shear modulus less than 3.5×10⁶ pounds per square inch or greater than 4.5×10⁶ pounds per square inch.

Illustratively, forceps arm superior incline angle 120 may comprise any angle greater than 90.0 degrees. In one or more embodiments, forceps arm superior incline angle 120 may comprise any angle in a range of 150.0 to 170.0 degrees, e.g., forceps arm superior incline angle 120 may comprise a 160.31 degree angle. Illustratively, forceps arm superior incline angle 120 may comprise an angle less than 150.0 degrees or greater than 170.0 degrees. In one or more embodiments, forceps arm inferior decline angle 125 may comprise any angle greater than 90.0 degrees. Illustratively, forceps arm inferior decline angle 125 may comprise any angle in a range of 140.0 to 160.0 degrees, e.g., forceps arm inferior decline angle 125 may comprise a 149.56 degree angle. In one or more embodiments, forceps arm inferior decline angle 125 may comprise an angle less than 140.0 degrees or greater than 160.0 degrees. Illustratively, forceps arm inferior decline angle 125 may comprise any angle less than forceps arm superior incline angle 120, e.g., forceps arm inferior decline angle 125 may comprise an angle in a range of 5.0 to 15.0 degrees less than forceps arm superior incline angle 120. In one or more embodiments, forceps arm inferior decline angle 125 may comprise an angle less than 5.0 degrees or greater than 15.0 degrees less than forceps arm superior incline angle 120.

Illustratively, forceps arm superior decline angle 130 may comprise any angle less than 90.0 degrees. In one or more embodiments, forceps arm superior decline angle 130 may comprise any angle in a range of 5.0 to 15.0 degrees, e.g., forceps arm superior decline angle 130 may comprise an 11.3 degree angle. Illustratively, forceps arm superior decline angle 130 may comprise an angle less than 5.0 degrees or greater than 15.0 degrees. In one or more embodiments, forceps arm inferior incline angle 135 may comprise any angle less than 90.0 degrees. Illustratively, forceps arm inferior incline angle 135 may comprise any angle in a range of 15.0 to 30.0 degrees, e.g., forceps arm inferior incline angle 135 may comprise a 23.08 degree angle. In one or more embodiments, forceps arm inferior incline angle 135 may comprise an angle less than 15.0 degrees or greater than 30.0 degrees. Illustratively, forceps arm inferior incline angle 135 may comprise any angle greater than forceps arm superior decline angle 130, e.g., forceps arm inferior incline angle 135 may comprise an angle in a range of 5.0 to 15.0 degrees greater than forceps arm superior decline angle 130. In one or more embodiments, forceps arm inferior incline angle 135 may comprise an angle less than 5.0 degrees or greater than 15.0 degrees greater than forceps arm superior decline angle 130.

FIG. 1C is a schematic diagram illustrating a cross-sectional view in a sagittal plane of a forceps arm 100. Illustratively, a forceps arm 100 may comprise an internal conduit 160, a superior coolant path 161, an inferior coolant path 162, an internal conduit distal end 163, an inferior coolant path proximal end 165, a superior coolant path proximal end 166, a superior conduit separation distance 171, an inferior conduit separation distance 172, and a distal conduit separation distance 173. In one or more embodiments, internal conduit 160 may be configured to facilitate a coolant circulation, e.g., internal conduit 160 may be configured to facilitate a coolant circulation within a portion of forceps arm 100. For example, a coolant circulation within internal conduit 160 may be configured to facilitate an active cooling of a portion of forceps arm 100. Illustratively, a coolant transfer machine may be configured to circulate a coolant through internal conduit 160, e.g., a coolant transfer machine may be configured to circulate a coolant through internal conduit 160 to provide active cooling of a portion of forceps arm 100. For example, a coolant transfer machine may be configured to circulate a coolant through internal conduit 160 to provide active cooling of conductor tip 110. In one or more embodiments, a portion of internal conduit 160 may be lined with a sleeve or tubing, e.g., a portion of internal conduit 160 may be lined with a sleeve or tubing to prevent a corrosion of the portion of internal conduit 160.

In one or more embodiments, a coolant may be a liquid or a gas, e.g., a coolant may comprise a heat transfer fluid or an inert gas. Illustratively, a coolant may be configured to transfer heat from a portion of forceps arm 100 without corrosion of a portion of internal conduit 160. In one or more embodiments, a coolant may be non-toxic and biocompatible. Illustratively, a coolant may be toxic and non-biocompatible. In one or more embodiments, a coolant may comprise a gas, e.g., a coolant may comprise air, hydrogen, helium, argon, sulfur hexafluoride, etc. Illustratively, a coolant may comprise a liquid, e.g., a coolant may comprise water, deionized water, saline, betaine, ethylene glycol, a combination of water and ethylene glycol, diethylene glycol, a combination of water and diethylene glycol, propylene glycol, a combination of water and propylene glycol, polyalkylene glycol, mineral oil, castor oil, silicone oil, fluorocarbon oil, transformer oil, a Freon, a refrigerant, carbon dioxide, liquid nitrogen, liquid hydrogen, etc. In one or more embodiments, a coolant may comprise a carrier liquid and a plurality of nanometersized particles, e.g., a nanofluid.

Illustratively, a coolant may comprise water and silver rods in a concentration range of 0.3 to 0.7 percent by volume wherein the silver rods have a diameter in a range of 40.0 to 70.0 nanometers and an average length in a range of 5.0 to 10.0 micrometers. In one or more embodiments, a coolant may comprise water and silver rods in a concentration of less than 0.3 percent by volume or greater than 0.7 percent by volume. Illustratively, a coolant may comprise water and silver rods having a diameter of less than 40.0 nanometers or greater than 70.0 nanometers. In one or more embodiments, a coolant may comprise water and silver rods having an average length of less than 5.0 micrometers or greater than 10.0 micrometers. Illustratively, a coolant may comprise ethylene glycol and silver rods in a concentration range of 0.3 to 0.7 percent by volume wherein the silver rods have a diameter in a range of 40.0 to 70.0 nanometers and an average length in a range of 5.0 to 10.0 micrometers. In one or more embodiments, a coolant may comprise ethylene glycol and silver rods in a concentration of less than 0.3 percent by volume or greater than 0.7 percent by volume. Illustratively, a coolant may comprise ethylene glycol and silver rods having a diameter of less than 40.0 nanometers or greater than 70.0 nanometers. In one or more embodiments, a coolant may comprise ethylene glycol and silver rods having an average length of less than 5.0 micrometers or greater than 10.0 micrometers.

Illustratively, a coolant may comprise water and copper rods in a concentration range of 0.3 to 0.7 percent by volume wherein the copper rods have a diameter in a range of 40.0 to 70.0 nanometers and an average length in a range of 5.0 to 10.0 micrometers. In one or more embodiments, a coolant may comprise water and copper rods in a concentration of less than 0.3 percent by volume or greater than 0.7 percent by volume. Illustratively, a coolant may comprise water and copper rods having a diameter of less than 40.0 nanometers or greater than 70.0 nanometers. In one or more embodiments, a coolant may comprise water and copper rods having an average length of less than 5.0 micrometers or greater than 10.0 micrometers. Illustratively, a coolant may comprise ethylene glycol and copper rods in a concentration range of 0.3 to 0.7 percent by volume wherein the copper rods have a diameter in a range of 40.0 to 70.0 nanometers and an average length in a range of 5.0 to 10.0 micrometers. In one or more embodiments, a coolant may comprise ethylene glycol and copper rods in a concentration of less than 0.3 percent by volume or greater than 0.7 percent by volume. Illustratively, a coolant may comprise ethylene glycol and copper rods having a diameter of less than 40.0 nanometers or greater than 70.0 nanometers. In one or more embodiments, a coolant may comprise ethylene glycol and copper rods having an average length of less than 5.0 micrometers or greater than 10.0 micrometers.

In one or more embodiments, a coolant transfer machine may be configured to pump a coolant into superior coolant path proximal end 166, e.g., a coolant transfer machine may be configured to pressurize a coolant wherein the coolant is configured to ingress internal conduit 160 at superior coolant path proximal end 166. Illustratively, a coolant transfer machine may be configured to circulate a coolant along superior coolant path 161, e.g., a coolant transfer machine may be configured to circulate a coolant along superior coolant path 161 towards internal conduit distal end 163. In one or more embodiments, a coolant transfer machine may be configured to pressurize a coolant wherein the coolant is configured to ingress internal conduit 160 along superior coolant path 161, e.g., a coolant transfer machine may be configured to pressurize a coolant wherein the coolant is configured to ingress internal conduit 160 along superior coolant path 161 and then egress internal conduit 160 along inferior coolant path 162. Illustratively, a coolant transfer machine may be configured to circulate a coolant through internal conduit 160 wherein the coolant ingresses superior coolant path proximal end 166 at a first coolant temperature and the coolant egresses inferior coolant path proximal end 165 at a second coolant temperature. In one or more embodiments, the second coolant temperature may be greater than the first coolant temperature. Illustratively, a coolant transfer machine may be configured to ingress a coolant into a portion of conductor tip 110, e.g., a coolant transfer machine may be configured to circulate a coolant along superior coolant path 161 into a portion of conductor tip 110. In one or more embodiments, a coolant transfer machine may be configured to egress a coolant out from a portion of conductor tip 110, e.g., a coolant transfer machine may be configured to circulate a coolant along inferior coolant path 162 out from a portion of conductor tip 110.

In one or more embodiments, a coolant transfer machine may be configured to pump a coolant into inferior coolant path proximal end 165, e.g., a coolant transfer machine may be configured to pressurize a coolant wherein the coolant is configured to ingress internal conduit 160 at inferior coolant path proximal end 165. Illustratively, a coolant transfer machine may be configured to circulate a coolant along inferior coolant path 162, e.g., a coolant transfer machine may be configured to circulate a coolant along inferior coolant path 162 towards internal conduit distal end 163. In one or more embodiments, a coolant transfer machine may be configured to pressurize a coolant wherein the coolant is configured to ingress internal conduit 160 along inferior coolant path 162, e.g., a coolant transfer machine may be configured to pressurize a coolant wherein the coolant is configured to ingress internal conduit 160 along inferior coolant path 162 and then egress internal conduit 160 along superior coolant path 161. Illustratively, a coolant transfer machine may be configured to circulate a coolant through internal conduit 160 wherein the coolant ingresses inferior coolant path proximal end 165 at a first coolant temperature and the coolant egresses superior coolant path proximal end 166 at a second coolant temperature. In one or more embodiments, the second coolant temperature may be greater than the first coolant temperature. Illustratively, a coolant transfer machine may be configured to ingress a coolant into a portion of conductor tip 110, e.g., a coolant transfer machine may be configured to circulate a coolant along inferior coolant path 162 into a portion of conductor tip 110. In one or more embodiments, a coolant transfer machine may be configured to egress a coolant out from a portion of conductor tip 110, e.g., a coolant transfer machine may be configured to circulate a coolant along superior coolant path 161 out from a portion of conductor tip 110.

Illustratively, a coolant transfer machine may be configured to ingress a coolant into a portion of conductor tip 110 at a first coolant temperature and egress the coolant out from the portion of conductor tip 110 at a second coolant temperature. In one or more embodiments, the second coolant temperature may be greater than the first coolant temperature. Illustratively, a coolant transfer machine may be configured to circulate a coolant through internal conduit 160 wherein a portion of conductor tip 110 has a first conductor tip temperature before the coolant ingresses the portion of conductor tip 110 and wherein the portion of conductor tip 110 has a second conductor tip temperature after the coolant egresses the portion of conductor tip 110. In one or more embodiments, the first conductor tip temperature may be greater than the second conductor tip temperature.

In one or more embodiments, superior conduit separation distance 171 may comprise a distance between a surface of conductor tip 110 and a superior side of internal conduit 160, e.g., superior conduit separation distance 171 may comprise a distance between a surface of conductor tip 110 and a superior side of superior coolant path 161. Illustratively, superior conduit separation distance 171 may comprise a distance in a range of 0.0045 to 0.0155 inches, e.g., superior conduit separation distance 171 may comprise a distance of 0.01 inches. In one or more embodiments, superior conduit separation distance 171 may comprise a distance of less than 0.0045 inches or greater than 0.0155 inches. Illustratively, inferior conduit separation distance 172 may comprise a distance between a surface of conductor tip 110 and an inferior side of internal conduit 160, e.g., inferior conduit separation distance 172 may comprise a distance between a surface of conductor tip 110 and an inferior side of inferior coolant path 162. In one or more embodiments, inferior conduit separation distance 172 may comprise a distance in a range of 0.0045 to 0.0155 inches, e.g., inferior conduit separation distance 172 may comprise a distance of 0.01 inches. Illustratively, inferior conduit separation distance 172 may comprise a distance of less than 0.0045 inches or greater than 0.0155 inches. In one or more embodiments, distal conduit separation distance 173 may comprise a distance between a surface of conductor tip 110 and internal conduit distal end 163, e.g., distal conduit separation distance 173 may comprise a distance between forceps arm distal end 101 and internal conduit distal end 163. Illustratively, distal conduit separation distance 173 may comprise a distance in a range of 0.073 inches to 0.1 inches, e.g., distal conduit separation distance 173 may comprise a distance of 0.087 inches. In one or more embodiments, distal conduit separation distance 173 may comprise a distance of less than 0.073 inches or greater than 0.1 inches.

FIG. 1D is a schematic diagram illustrating a superior view of a forceps arm 100. FIG. 1E is a schematic diagram illustrating a cross-sectional view in a traverse plane of a forceps arm 100. Illustratively, a forceps arm 100 may comprise an internal conduit 160 having a first deviation in sagittal plane 167, a second deviation in sagittal plane 168, a third deviation in sagittal plane 169, and a fourth deviation in sagittal plane 164. In one or more embodiments, forceps arm 100 may be manufactured by additive manufacturing, e.g., forceps arm 100 may be manufactured by selective laser melting, direct metal laser sintering, selective laser sintering, fused deposition modeling, fused filament fabrication, stereolithography, laminated object manufacturing, etc. Illustratively, forceps arm 100 may be manufactured from one or more components, e.g., forceps arm 100 may be manufactured in one or more frontal plane layers wherein each layer of the one or more layers may comprise a portion of internal conduit 160. In one more embodiments, forceps arm 100 may be manufactured in one or more sagittal plane layers wherein each layer of the one or more layers may comprise a portion of internal conduit 160. Illustratively, forceps arm 100 may be manufactured in one or more transverse plane layers wherein each layer of the one or more layers may comprise a portion of internal conduit 160. In one or more embodiments, a first portion of forceps arm 100 may be manufactured by additive manufacturing and a second portion of forceps arm 100 may be manufactured by computer numerical control machining, e.g., conductor tip 110 may be manufactured by additive manufacturing and a portion of forceps arm 100 other than conductor tip 110 may be manufactured by computer numerical control machining. Illustratively, internal conduit 160 may be manufactured by fabricating a channel into a substrate, e.g., by machining, cutting, etching, etc., and then sealing the channel to form internal conduit 160. In one or more embodiments, internal conduit 160 may be manufactured by drilling into a substrate. Illustratively, forceps arm 100 may be manufactured by a mold, e.g., an injection mold.

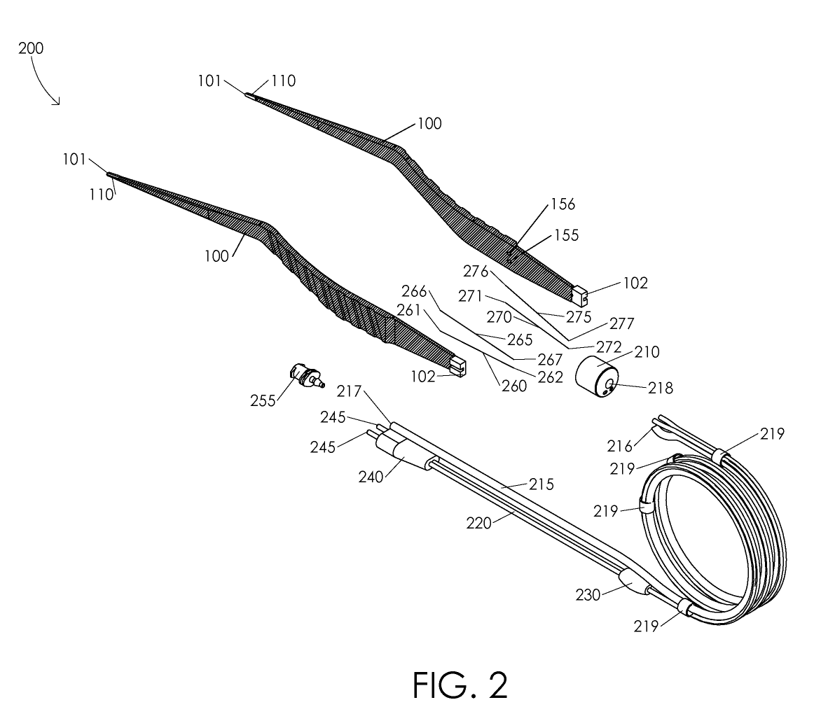

FIG. 2 is a schematic diagram illustrating an exploded view of a bipolar forceps with active cooling assembly 200. In one or more embodiments, a bipolar forceps with active cooling assembly 200 may comprise a pair of forceps arms 100, a coolant multiplexer 210, a coolant transfer tube 215, a coolant transfer tube housing 218, a fastener 219, a bipolar cord 220, a bipolar cord separation control 230, an electrosurgical generator adapter 240, an electrosurgical generator interface 245, a coolant transfer machine interface 255, a first inferior coolant path interface 260, a first superior coolant path interface 265, a second inferior coolant path interface 270, and a second superior coolant path interface 275. In one or more embodiments, one or more fasteners 219 may be configured to fasten coolant transfer tube 215 to bipolar cord 220, e.g., one or more fasteners 219 may be configured to fasten bipolar cord 220 to coolant transfer tube 215. Illustratively, a portion of each forceps arm 100 may be coated with a material having a high u) electrical resistivity, e.g., a portion of each forceps arm 100 may be coated with an electrical insulator material. In one or more embodiments, input conductor housings 103 and conductor tips 110 may not be coated with a material, e.g., input conductor housings 103 and conductor tips 110 may comprise electrical leads. In one or more embodiments, a portion of each forceps arm 100 may be anodized to increase a thickness of a natural oxide layer on a surface of each forceps arm 100, e.g., a portion of each forceps arm 100 may be coated with an oxide layer greater than 15.0 nanometers. Illustratively, each forceps arm 100 may be subjected to an electrolytic passivation process configured to increase a thickness of an oxide layer on the surface of each forceps arm 100, e.g., a portion of each forceps arm may be coated with an oxide layer greater than 15.0 nanometers. In one or more embodiments, a portion of each forceps arm 100 may be coated with an oxide layer having a thickness in a range of 15.0 nanometers to 1.0 micrometers. Illustratively, a portion of each forceps arm 100 may be coated with an oxide layer having a thickness less than 15.0 nanometers or greater than 1.0 micrometers. In one or more embodiments, an oxide layer on a surface of each forceps arm 100 may be the only electrical insulator material on the surface of each forceps arm 100, e.g., an oxide layer may be the only electrical insulation on a portion of each forceps arm 100.

Illustratively, a portion of each forceps arm 100 may be coated with a thermoplastic material, e.g., a portion of each forceps arm 100 may be coated with nylon. In one or more embodiments, a portion of each forceps arm 100 may be coated with a fluoropolymer, e.g., a portion of each forceps arm 100 may be coated with polyvinylidene fluoride.

Illustratively, a portion of each forceps arm 100 may be coated with a material having an electrical conductivity less than 1.0×10⁻⁸ Siemens per meter at a temperature of 20.0° C., e.g., a portion of each forceps arm 100 may be coated with a material having an electrical conductivity of 1.0×10⁻¹² Siemens per meter at a temperature of 20.0° C. In one or more embodiments, a portion of each forceps arm 100 may be coated with a material having a thermal conductivity of less than 1.0 Watts per meter Kelvin at a temperature of 20.0° C., e.g., a portion of each forceps arm 100 may be coated with a material having a thermal conductivity of 0.25 Watts per meter Kelvin at a temperature of 20.0° C. Illustratively, a portion of each forceps arm 100 may be coated with a material having an electrical conductivity of less than 1.0×10⁻⁸ Siemens per meter and a thermal conductivity of less than 1.0 Watts per meter Kelvin at a temperature of 20.0° C., e.g., a portion of each forceps arm 100 may be coated with a material having an electrical conductivity of 1.0×10⁻¹² Siemens per meter and a thermal conductivity of 0.25 Watts per meter Kelvin at a temperature of 20.0° C. In one or more embodiments, a portion of each forceps arm 100 may be coated with a material wherein a coating thickness of the material is in a range of 0.005 to 0.008 inches, e.g., a portion of each forceps arm 100 may be coated with a material wherein a coating thickness of the material is 0.0065 inches. Illustratively, a portion of each forceps arm 100 may be coated with a material wherein a coating thickness of the material is less than 0.005 inches or greater than 0.008 inches. In one or more embodiments, a portion of each forceps arm 100 may be coated with a material having an electrical conductivity of less than 1.0×10⁻⁸ Siemens per meter and a thermal conductivity of less than 1.0 Watts per meter Kelvin at a temperature of 20.0° C. wherein a coating thickness of the material is in a range of 0.005 to 0.008 inches, e.g., a portion of each forceps arm 100 may be coated with a material having an electrical conductivity of 1.0×10⁻¹² Siemens per meter and a thermal conductivity of 0.25 Watts per meter Kelvin at a temperature of 20.0° C. wherein a coating thickness of the material is 0.0065 inches. Illustratively, a portion of each forceps arm 100 may be coated with a material having a material mass in a range of 0.0015 to 0.0025 pounds, e.g., a portion of each forceps arm 100 may be coated with a material having a material mass of 0.0021 pounds. In one or more embodiments, a portion of each forceps arm 100 may be coated with a material having a material mass less than 0.0015 pounds or greater than 0.0025 pounds.

Illustratively, coolant multiplexer 210 may comprise a first forceps arm housing and a second forceps arm housing. In one or more embodiments, coolant multiplexer 210 may be configured to separate a first bipolar input conductor and a second bipolar input conductor, e.g., coolant multiplexer 210 comprise a material with an electrical resistivity greater than 1×10¹⁶ ohm meters. Illustratively, coolant multiplexer 210 may comprise a material with an electrical resistivity less than or equal to 1×10¹⁶ ohm meters. In one or more embodiments, coolant multiplexer 210 may comprise an interface between bipolar cord 220 and forceps arms 100. Illustratively, a first bipolar input conductor and a second bipolar input conductor may be disposed within bipolar cord 220, e.g., bipolar cord 220 may be configured to separate the first bipolar input conductor and the second bipolar input conductor. In one or more embodiments, a first bipolar input conductor may be electrically connected to first forceps arm 100, e.g., the first bipolar input conductor may be disposed within input conductor housing 103. Illustratively, a second bipolar input conductor may be electrically connected to second forceps arm 100, e.g., the second bipolar input conductor may be disposed within input conductor housing 103. In one or more embodiments, a portion of first forceps arm 100 may be disposed within a first forceps arm housing, e.g., first forceps arm proximal end 102 may be disposed within a first forceps arm housing. Illustratively, first forceps arm 100 may be fixed within a first forceps arm housing, e.g., by an adhesive or any suitable fixation means. In one or more embodiments, a first bipolar input conductor may be disposed within a first forceps arm housing, e.g., the first bipolar input conductor may be electrically connected to first forceps arm 100. Illustratively, a first bipolar input conductor may be fixed within a first forceps arm housing wherein the first bipolar input conductor is electrically connected to first forceps arm 100. In one or more embodiments, a portion of second forceps arm 100 may be disposed within a second forceps arm housing, e.g., second forceps arm proximal end 102 may be disposed within a second forceps arm housing. Illustratively, second forceps arm 100 may be fixed within a second forceps arm housing, e.g., by an adhesive or any suitable fixation means. In one or more embodiments, a second bipolar input conductor may be disposed within a second forceps arm housing, e.g., the second bipolar input conductor may be electrically connected to second forceps arm 100. Illustratively, a second bipolar input conductor may be fixed within a second forceps arm housing wherein the second bipolar input conductor is electrically connected to second forceps arm 100.

In one or more embodiments, electrosurgical generator adaptor 240 may comprise a first electrosurgical generator interface 245 and a second electrosurgical generator interface 245. Illustratively, first electrosurgical generator interface 245 and second electrosurgical generator interface 245 may be configured to connect to an electrosurgical generator. In one or more embodiments, connecting first electrosurgical generator interface 245 and second electrosurgical generator interface 245 to an electrosurgical generator may be configured to electrically connect a first bipolar input conductor to a first electrosurgical generator output and to electrically connect a second bipolar input conductor to a second electrosurgical generator output. Illustratively, connecting a first bipolar input conductor to a first electrosurgical generator output may be configured to electrically connect first forceps arm 100 to the first electrosurgical generator output. In one or more embodiments, connecting a second bipolar input conductor to a second electrosurgical generator output may be configured to electrically connect second forceps arm 100 to the second electrosurgical generator output.

Illustratively, forceps arms 100 may be fixed within forceps arm housings wherein forceps arm proximal ends 102 are fixed within coolant multiplexer 210 and forceps arm distal ends 101 are separated by a maximum conductor tip 110 separation distance. In one or more embodiments, a surgeon may decrease a distance between first forceps arm distal end 101 and second forceps arm distal end 101, e.g., by applying a force to a lateral portion of forceps arms 100. Illustratively, a surgeon may decrease a distance between first forceps arm distal end 101 and second forceps arm distal end 101, e.g., until first forceps arm distal end 101 contacts second forceps arm distal end 101. In one or more embodiments, a contact between first forceps arm distal end 101 and second forceps arm distal end 101 may be configured to electrically connect conductor tips 110. Illustratively, an electrical connection of conductor tips 110 may be configured to close an electrical circuit. In one or more embodiments, a surgeon may increase a distance between first forceps arm distal end 101 and second forceps arm distal end 101, e.g., by reducing a force applied to a lateral portion of forceps arms 100. Illustratively, increasing a distance between first forceps arm distal end 101 and second forceps arm distal end 101 may be configured to separate conductor tips 110. In one or more embodiments, a separation of conductor tips 110 may be configured to open an electrical circuit.

Illustratively, coolant transfer tube 215 may comprise a coolant transfer tube distal end 216 and a coolant transfer tube proximal end 217. In one or more embodiments, coolant transfer machine interface 255 may be configured to interface with a coolant transfer machine to facilitate a supply of a coolant from the coolant transfer machine to coolant transfer tube 215. Illustratively, coolant transfer machine interface 255 may be configured to interface with a coolant transfer machine to facilitate a return of a coolant to the coolant transfer machine from the coolant transfer tube 215. In one or more embodiments, coolant transfer machine interface 255 may be configured to interface with a coolant transfer machine to simultaneously facilitate a supply of a first coolant from the coolant transfer machine to coolant transfer tube 215 and a return of a second coolant to the coolant transfer machine from the coolant transfer tube 215. Illustratively, a portion of coolant transfer machine interface 255 may be disposed within a portion of coolant transfer tube 215, e.g., a portion of coolant transfer machine interface 255 may be disposed in coolant transfer tube proximal end 217. In one or more embodiments, a portion of coolant transfer tube 215 may be disposed within a portion of coolant multiplexer 210, e.g., coolant transfer tube distal end 216 may be disposed within coolant transfer tube housing 218.

Illustratively, coolant transfer tube 215 may comprise a thermally insulated portion configured to thermally insulate a first coolant from a second coolant within coolant transfer tube 215. In one or more embodiments, a coolant being transferred to internal conduit 160 may have a first coolant temperature and a coolant being transferred from internal conduit 160 may have a second coolant temperature. Illustratively, the second coolant temperature may be greater than the first coolant temperature. In one or more embodiments, coolant transfer tube 215 may comprise a thermally insulated portion configured to thermally insulate a coolant being transferred to internal conduit 160 from a coolant being transferred from internal conduit 160. Illustratively, a coolant temperature of a coolant may be lowered before transferring the coolant to internal conduit 160, e.g., a coolant may be stored in a refrigerated environment to improve heat transfer within internal conduit 160. In one or more embodiments, coolant transfer tube 215 may comprise a thermally insulated portion configured to thermally insulate a first coolant and a second coolant from a third coolant and a fourth coolant within coolant transfer tube 215. Illustratively, a first coolant being transferred to an internal conduit 160 of a first forceps arm 100 may have a first coolant temperature, a second coolant being transferred to an internal conduit 160 of a second forceps arm 100 may have a second coolant temperature, a third coolant being transferred from the internal conduit 160 of the first forceps arm 100 may have a third coolant temperature, and a fourth coolant being transferred from the internal conduit 160 of the second forceps arm 100 may have a fourth coolant temperature. In one or more embodiments, the first coolant temperature may be less than the third coolant temperature and less than the fourth coolant temperature. Illustratively, the second coolant temperature may be less than the third coolant temperature and less than the fourth coolant temperature. In one or more embodiments, coolant transfer tube 215 may comprise a thermally insulated portion configured to thermally insulate a first coolant being transferred to a first internal conduit 160 and a second coolant being transferred to a second internal conduit 160 from a third coolant being transferred from the first internal conduit 160 and a fourth coolant being transferred from the second internal conduit 160.

Illustratively, first inferior coolant path interface 260 may comprise a first inferior coolant path interface distal end 261 and a first inferior coolant path proximal end 262. In one or more embodiments, a portion of first inferior coolant path interface 260 may be disposed in coolant multiplexer 210, e.g., first inferior coolant path proximal end 262 may be disposed in coolant multiplexer 210. Illustratively, a portion of first inferior coolant path interface 260 may be disposed in an internal conduit 160 of a first forceps arm 100, e.g., first inferior coolant path distal end 261 may be disposed in an inferior coolant path proximal end 165 of a first forceps arm 100. In one or more embodiments, first inferior coolant path interface 260 may be configured to facilitate a transfer of a coolant. Illustratively, first inferior coolant path interface 260 may be configured to transfer a coolant from coolant transfer tube 215 to an inferior coolant path 162 of a first forceps arm 100. In one or more embodiments, first inferior coolant path interface 260 may be configured to transfer a coolant from an inferior coolant path 162 of a first forceps arm 100 to coolant transfer tube 215.

Illustratively, first superior coolant path interface 265 may comprise a first superior coolant path interface distal end 266 and a first superior coolant path proximal end 267. In one or more embodiments, a portion of first superior coolant path interface 265 may be disposed in coolant multiplexer 210, e.g., first superior coolant path proximal end 267 may be disposed in coolant multiplexer 210. Illustratively, a portion of first superior coolant path interface 265 may be disposed in an internal conduit 160 of a first forceps arm 100, e.g., first superior coolant path distal end 266 may be disposed in a superior coolant path proximal end 166 of a first forceps arm 100. In one or more embodiments, first superior coolant path interface 265 may be configured to facilitate a transfer of a coolant. Illustratively, first superior coolant path interface 265 may be configured to transfer a coolant from coolant transfer tube 215 to a superior coolant path 161 of a first forceps arm 100. In one or more embodiments, first superior coolant path interface 265 may be configured to transfer a coolant from a superior coolant path 161 of a first forceps arm 100 to coolant transfer tube 215.

Illustratively, second inferior coolant path interface 270 may comprise a second inferior coolant path interface distal end 271 and a second inferior coolant path proximal end 272. In one or more embodiments, a portion of second inferior coolant path interface 270 may be disposed in coolant multiplexer 210, e.g., second inferior coolant path proximal end 272 may be disposed in coolant multiplexer 210. Illustratively, a portion of second inferior coolant path interface 270 may be disposed in an internal conduit 160 of a second forceps arm 100, e.g., second inferior coolant path distal end 271 may be disposed in an inferior coolant path proximal end 165 of a second forceps arm 100. In one or more embodiments, second inferior coolant path interface 270 may be configured to facilitate a transfer of a coolant. Illustratively, second inferior coolant path interface 270 may be configured to transfer a coolant from coolant transfer tube 215 to an inferior coolant path 162 of a second forceps arm 100. In one or more embodiments, second inferior coolant path interface 270 may be configured to transfer a coolant from an inferior coolant path 162 of a second forceps arm 100 to coolant transfer tube 215.

Illustratively, second superior coolant path interface 275 may comprise a second superior coolant path interface distal end 276 and a second superior coolant path proximal end 277. In one or more embodiments, a portion of second superior coolant path interface 275 may be disposed in coolant multiplexer 210, e.g., second superior coolant path proximal end 277 may be disposed in coolant multiplexer 210. Illustratively, a portion of second superior coolant path interface 275 may be disposed in an internal conduit 160 of a second forceps arm 100, e.g., second superior coolant path distal end 276 may be disposed in a superior coolant path proximal end 166 of a second forceps arm 100. In one or more embodiments, second superior coolant path interface 275 may be configured to facilitate a transfer of a coolant. Illustratively, second superior coolant path interface 275 may be configured to transfer a coolant from coolant transfer tube 215 to a superior coolant path 161 of a second forceps arm 100. In one or more embodiments, second superior coolant path interface 275 may be configured to transfer a coolant from a superior coolant path 161 of a second forceps arm 100 to coolant transfer tube 215.

In one or more embodiments, coolant multiplexer 210 may be configured to connect first inferior coolant path interface 260 to a coolant supply line of coolant transfer tube 215, e.g., coolant multiplexer 210 may be configured to connect first inferior coolant path interface proximal end 262 to a coolant supply line of coolant transfer tube 215. Illustratively, coolant multiplexer 210 may be configured to connect first inferior coolant path interface 260 to a coolant return line of coolant transfer tube 215, e.g., coolant multiplexer 210 may be configured to connect first inferior coolant path interface proximal end 262 to a coolant return line of coolant transfer tube 215. In one or more embodiments, coolant multiplexer 210 may be configured to connect first superior coolant path interface 265 to a coolant supply line of coolant transfer tube 215, e.g., coolant multiplexer 210 may be configured to connect first superior coolant path interface proximal end 267 to a coolant supply line of coolant transfer tube 215. Illustratively, coolant multiplexer 210 may be configured to connect first superior coolant path interface 265 to a coolant return line of coolant transfer tube 215, e.g., coolant multiplexer 210 may be configured to connect first superior coolant path interface proximal end 267 to a coolant return line of coolant transfer tube 215. In one or more embodiments, coolant multiplexer 210 may be configured to connect second inferior coolant path interface 270 to a coolant supply line of coolant transfer tube 215, e.g., coolant multiplexer 210 may be configured to connect second inferior coolant path interface proximal end 272 to a coolant supply line of coolant transfer tube 215. Illustratively, coolant multiplexer 210 may be configured to connect second inferior coolant path interface 270 to a coolant return line of coolant transfer tube 215, e.g., coolant multiplexer 210 may be configured to connect second inferior coolant path interface proximal end 272 to a coolant return line of coolant transfer tube 215. In one or more embodiments, coolant multiplexer 210 may be configured to connect second superior coolant path interface 275 to a coolant supply line of coolant transfer tube 215, e.g., coolant multiplexer 210 may be configured to connect second superior coolant path interface proximal end 277 to a coolant supply line of coolant transfer tube 215. Illustratively, coolant multiplexer 210 may be configured to connect second superior coolant path interface 275 to a coolant return line of coolant transfer tube 215, e.g., coolant multiplexer 210 may be configured to connect second superior coolant path interface proximal end 277 to a coolant return line of coolant transfer tube 215.

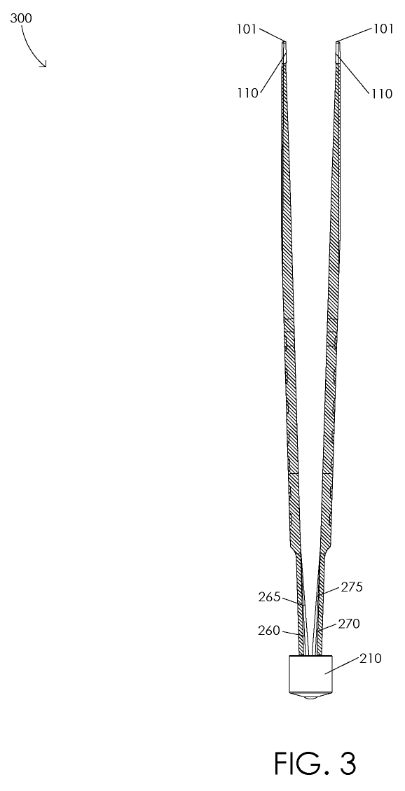

FIG. 3 is a schematic diagram illustrating an assembled bipolar forceps with active cooling 300. In one or more embodiments, an assembled bipolar forceps with active cooling 300 may be configured to decrease a temperature of conductor tips 110 by circulating a coolant through an internal conduit 160 of a first forceps arm 100 and by circulating a coolant through an internal conduit 160 of a second forceps arm 100. Illustratively, coolant transfer machine interface 255 may be configured to interface with a coolant transfer machine to circulate a coolant into an internal conduit 160 of a first forceps arm 100, e.g., coolant transfer machine interface 255 may be configured to interface with a coolant transfer machine to circulate a coolant into a superior coolant path proximal end 166 of a first forceps arm 100. In one or more embodiments, coolant transfer machine interface 255 may be configured to interface with a coolant transfer machine to circulate a coolant through a superior coolant path 161 of a first forceps arm 100. Illustratively, coolant transfer machine interface 255 may be configured to interface with a coolant transfer machine to circulate a coolant into a conductor tip 110 of a first forceps arm 100. In one or more embodiments, coolant transfer machine interface 255 may be configured to interface with a coolant transfer machine to circulate a coolant out from a conductor tip 110 of a first forceps arm 100. Illustratively, coolant transfer machine interface 255 may be configured to interface with a coolant transfer machine to circulate a coolant through an inferior coolant path 162 of a first forceps arm 100. In one or more embodiments, coolant transfer machine interface 255 may be configured to interface with a coolant transfer machine to circulate a coolant out from an internal conduit 160 of a first forceps arm 100, e.g., coolant transfer machine interface 255 may be configured to interface with a coolant transfer machine to circulate a coolant out from an inferior coolant path proximal end 165 of a first forceps arm 100.

Illustratively, coolant transfer machine interface 255 may be configured to interface with a coolant transfer machine to circulate a coolant into an internal conduit 160 of a first forceps arm 100, e.g., coolant transfer machine interface 255 may be configured to interface with a coolant transfer machine to circulate a coolant into an inferior coolant path proximal end 165 of a first forceps arm 100. In one or more embodiments, coolant transfer machine interface 255 may be configured to interface with a coolant transfer machine to circulate a coolant through an inferior coolant path 162 of a first forceps arm 100. Illustratively, coolant transfer machine interface 255 may be configured to interface with a coolant transfer machine to circulate a coolant into a conductor tip 110 of a first forceps arm 100. In one or more embodiments, coolant transfer machine interface 255 may be configured to interface with a coolant transfer machine to circulate a coolant out from a conductor tip 110 of a first forceps arm 100. Illustratively, coolant transfer machine interface 255 may be configured to interface with a coolant transfer machine to circulate a coolant through a superior coolant path 161 of a first forceps arm 100. In one or more embodiments, coolant transfer machine interface 255 may be configured to intern face with a coolant transfer machine to circulate a coolant out from an internal conduit 160 of a first forceps arm 100, e.g., coolant transfer machine interface 255 may be configured to interface with a coolant transfer machine to circulate a coolant out from a superior coolant path proximal end 166 of a first forceps arm 100.

Illustratively, coolant transfer machine interface 255 may be configured to interface with a coolant transfer machine to circulate a coolant into an internal conduit 160 of a second forceps arm 100, e.g., coolant transfer machine interface 255 may be configured to interface with a coolant transfer machine to circulate a coolant into a superior coolant path proximal end 166 of a second forceps arm 100. In one or more embodiments, coolant transfer machine interface 255 may be configured to interface with a coolant transfer machine to circulate a coolant through a superior coolant path 161 of a second forceps arm 100. Illustratively, coolant transfer machine interface 255 may be configured to interface with a coolant transfer machine to circulate a coolant into a conductor tip 110 of a second forceps arm 100. In one or more embodiments, coolant transfer machine interface 255 may be configured to interface with a coolant transfer machine to circulate a coolant out from a conductor tip 110 of a second forceps arm 100. Illustratively, coolant transfer machine interface 255 may be configured to interface with a coolant transfer machine to circulate a coolant through an inferior coolant path 162 of a second forceps arm 100. In one or more embodiments, coolant transfer machine interface 255 may be configured to interface with a coolant transfer machine to circulate a coolant out from an internal conduit 160 of a second forceps arm 100, e.g., coolant transfer machine interface 255 may be configured to interface with a coolant transfer machine to circulate a coolant out from an inferior coolant path proximal end 165 of a second forceps arm 100.

Illustratively, coolant transfer machine interface 255 may be configured to interface with a coolant transfer machine to circulate a coolant into an internal conduit 160 of a second forceps arm 100, e.g., coolant transfer machine interface 255 may be configured to interface with a coolant transfer machine to circulate a coolant into an inferior coolant path proximal end 165 of a second forceps arm 100. In one or more embodiments, coolant transfer machine interface 255 may be configured to interface with a coolant transfer machine to circulate a coolant through an inferior coolant path 162 of a second forceps arm 100. Illustratively, coolant transfer machine interface 255 may be configured to interface with a coolant transfer machine to circulate a coolant into a conductor tip 110 of a second forceps arm 100. In one or more embodiments, coolant transfer machine interface 255 may be configured to interface with a coolant transfer machine to circulate a coolant out from a conductor tip 110 of a second forceps arm 100. Illustratively, coolant transfer machine interface 255 may be configured to interface with a coolant transfer machine to circulate a coolant through a superior coolant path 161 of a second forceps arm 100. In one or more embodiments, coolant transfer machine interface 255 may be configured to interface with a coolant transfer machine to circulate a coolant out from an internal conduit 160 of a second forceps arm 100, e.g., coolant transfer machine interface 255 may be configured to interface with a coolant transfer machine to circulate a coolant out from a superior coolant path proximal end 166 of a second forceps arm 100.

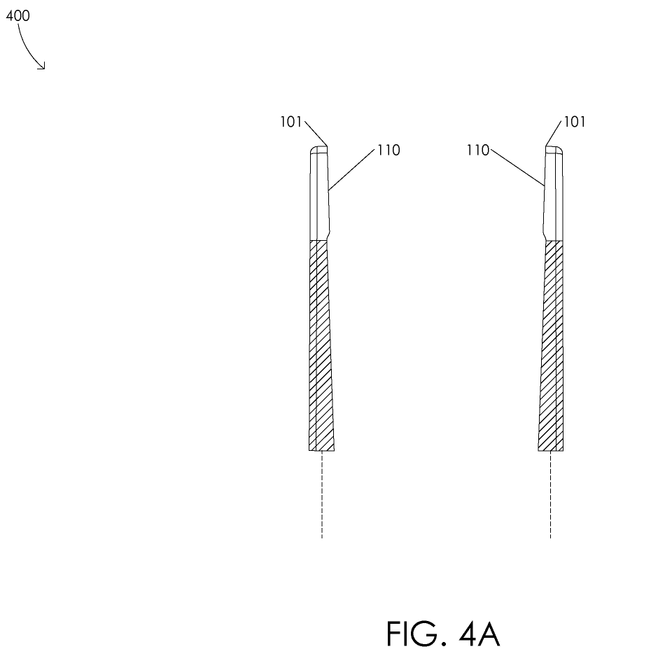

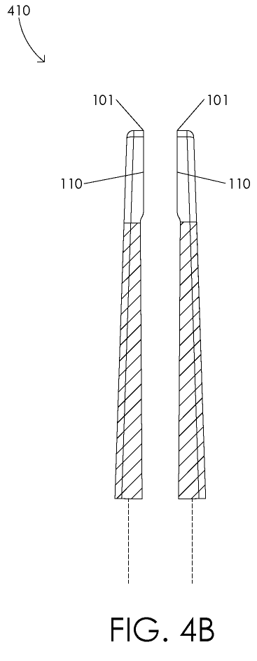

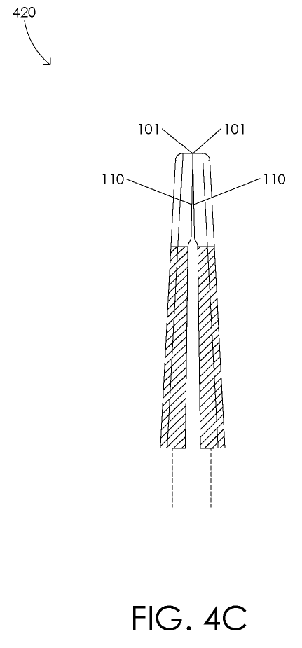

FIGS. 4A, 4B, and 4C are schematic diagrams illustrating a gradual closing of a bipolar forceps with active cooling. FIG. 4A illustrates conductor tips in an open orientation 400. Illustratively, conductor tips 110 may comprise conductor tips in an open orientation 400, e.g., when forceps arm distal ends 101 are separated by a maximum conductor tip 110 separation distance. In one or more embodiments, forceps arm distal ends 101 may be separated by a distance in a range of 0.5 to 0.7 inches when conductor tips 110 comprise conductor tips in an open orientation 400, e.g., forceps arm distal ends 101 may be separated by a distance of 0.625 inches when conductor tips 110 comprise conductor tips in an open orientation 400. Illustratively, forceps arm distal ends 101 may be separated by a distance less than 0.5 inches or greater than 0.7 inches when conductor tips 110 comprise conductor tips in an open orientation 400. In one or more embodiments, conductor tips 110 may comprise conductor tips in an open orientation 400, e.g., when no force is applied to a lateral portion of forceps arms 100.

FIG. 4B illustrates conductor tips in a partially closed orientation 410. Illustratively, an application of a force to a lateral portion of forceps arms 100 may be configured to gradually close conductor tips 110 from conductor tips in an open orientation 400 to conductor tips in a partially closed orientation 410. In one or more embodiments, an application of a force to a lateral portion of forceps arms 100 may be configured to decrease a distance between first forceps arm distal end 101 and second forceps arm distal end 101. Illustratively, an application of a force having a magnitude in a range of 0.05 to 0.3 pounds to a lateral portion of forceps arms 100 may be configured to decrease a distance between first forceps arm distal end 101 and second forceps arm distal end 101, e.g., an application of a force having a magnitude of 0.2 pounds to a lateral portion of forceps arms 100 may be configured to decrease a distance between first forceps arm distal end 101 and second forceps arm distal end 101. In one or more embodiments, an application of a force having a magnitude less than 0.05 pounds or greater than 0.3 pounds to a lateral portion of forceps arms 100 may be configured to decrease a distance between first forceps arm distal end 101 and second forceps arm distal end 101. Illustratively, a decrease of a distance between first forceps arm distal end 101 and second forceps arm distal end 101 may be configured to decrease a distance between conductor tips 110. In one or more embodiments, an application of a force having a magnitude in a range of 0.05 to 0.3 pounds to a lateral portion of forceps arms 100 may be configured to gradually close conductor tips 110 from conductor tips in an open orientation 400 to conductor tips in a partially closed orientation 410. Illustratively, an application of a force having a magnitude less than 0.05 pounds or greater than 0.3 pounds to a lateral portion of forceps arms 100 may be configured to gradually close conductor tips 110 from conductor tips in an open orientation 400 to conductor tips in a partially closed orientation 410. In one or more embodiments, an amount of force applied to a lateral portion of forceps arms 100 configured to close conductor tips 110 to conductor tips in a partially closed orientation 410 and a total mass of a bipolar forceps with active cooling may have a force applied to total mass ratio in a range of 1.25 to 8.75, e.g., an amount of force applied to a lateral portion of forceps arms 100 configured to close conductor tips 110 to conductor tips in a partially closed orientation 410 and a total mass of a bipolar forceps with active cooling may have a force applied to total mass ratio of 5.25. Illustratively, an amount of force applied to a lateral portion of forceps arms 100 configured to close conductor tips 110 to conductor tips in a partially closed orientation 410 and a total mass of a bipolar forceps with active cooling may have a force applied to total mass ratio less than 1.25 or greater than 8.75.

In one or more embodiments, a surgeon may dispose a tissue between a first forceps arm conductor tip 110 and a second forceps arm conductor tip 110, e.g., a surgeon may dispose a tumor tissue between a first forceps arm conductor tip 110 and a second forceps arm conductor tip 110. Illustratively, disposing a tissue between a first forceps arm conductor tip 110 and a second forceps arm conductor tip 110 may be configured to electrically connect the first forceps arm conductor tip 110 and the second forceps arm conductor tip 110, e.g., the tissue may electrically connect the first forceps arm conductor tip 110 and the second forceps arm conductor tip 110. In one or more embodiments, electrically connecting a first forceps arm conductor tip 110 and a second forceps arm conductor tip 110 may be configured to supply an electrical current to a tissue. Illustratively, supplying an electrical current to a tissue may be configured to coagulate the tissue, cauterize the tissue, ablate the tissue, etc. In one or more embodiments, electrically connecting a first forceps arm conductor tip 110 and a second forceps arm conductor tip 110 may be configured to seal a vessel, induce hemostasis, etc.

Illustratively, coagulating a tissue, cauterizing a tissue, ablating a tissue, sealing a vessel, or inducing hemostasis may be configured to increase a temperature of a first conductor tip 110. Increasing a temperature of a first conductor tip 110 may facilitate thermal spread to non-target tissue, e.g., increasing a temperature of a first conductor tip 110 may facilitate thermal spread to healthy tissue. In one or more embodiments, increasing a temperature of a first conductor tip 110 may be configured to cause a tissue to stick to the first conductor tip 110. Illustratively, decreasing a temperature of a first conductor tip 110 may be configured to prevent a tissue from sticking to the first conductor tip 110. In one or more embodiments, circulating a coolant through an internal conduit 160 of a first forceps arm 100 may be configured to decrease a temperature of a first conductor tip 110, e.g., circulating a coolant through an internal conduit 160 of a first forceps arm 100 may be configured to prevent a tissue from sticking to a first conductor tip 110. Illustratively, circulating a coolant through a first conductor tip 110 may be configured to decrease a temperature of the first conductor tip 110, e.g., circulating a coolant through a first conductor tip 110 may be configured to prevent a tissue from sticking to the first conductor tip 110. In one or more embodiments, circulating a coolant into a first conductor tip 110 and out from the first conductor tip 110 may be configured to decrease a temperature of the first conductor tip 110, e.g., circulating a coolant into a first conductor tip 110 and out from the first conductor tip 110 may be configured to prevent a tissue from sticking to the first conductor tip 110. Illustratively, circulating a coolant into a first conductor tip 110 and out from the first conductor tip 110 may be configured to increase a temperature of the coolant and decrease a temperature of the first conductor tip 110. In one or more embodiments, decreasing a temperature of a first conductor tip 110 may be configured to prevent thermal spread to a non-target tissue, e.g., decreasing a temperature of a first conductor tip 110 may be configured to prevent thermal spread to healthy tissue.

Illustratively, coagulating a tissue, cauterizing a tissue, ablating a tissue, sealing a vessel, or inducing hemostasis may be configured to increase a temperature of a second conductor tip 110. Increasing a temperature of a second conductor tip 110 may facilitate thermal spread to non-target tissue, e.g., increasing a temperature of a second conductor tip 110 may facilitate thermal spread to healthy tissue. In one or more embodiments, increasing a temperature of a second conductor tip 110 may be configured to cause a tissue to stick to the second conductor tip 110. Illustratively, decreasing a temperature of a second conductor tip 110 may be configured to prevent a tissue from sticking to the second conductor tip 110. In one or more embodiments, circulating a coolant through an internal conduit 160 of a second forceps arm 100 may be configured to decrease a temperature of a second conductor tip 110, e.g., circulating a coolant through an internal conduit 160 of a second forceps arm 100 may be configured to prevent a tissue from sticking to a second conductor tip 110. Illustratively, circulating a coolant through a second conductor tip 110 may be configured to decrease a temperature of the second conductor tip 110, e.g., circulating a coolant through a second conductor tip 110 may be configured to prevent a tissue from sticking to the second conductor tip 110. In one or more embodiments, circulating a coolant into a second conductor tip 110 and out from the second conductor tip 110 may be configured to decrease a temperature of the second conductor tip 110, e.g., circulating a coolant into a second conductor tip 110 and out from the second conductor tip 110 may be configured to prevent a tissue from sticking to the second conductor tip 110. Illustratively, circulating a coolant into a second conductor tip 110 and out from the second conductor tip 110 may be configured to increase a temperature of the coolant and decrease a temperature of the second conductor tip 110. In one or more embodiments, decreasing a temperature of a second conductor tip 110 may be configured to prevent thermal spread to a non-target tissue, e.g., decreasing a temperature of a second conductor tip 110 may be configured to prevent thermal spread to healthy tissue.

Illustratively, coagulating a tissue, cauterizing a tissue, ablating a tissue, sealing a vessel, or inducing hemostasis may be configured to increase a temperature of a first conductor tip 110 and increase a temperature of a second conductor tip 110. Increasing a temperature of a first conductor tip 110 and increasing a temperature of a second conducts for tip 110 may facilitate thermal spread to non-target tissue, e.g., increasing a temperature of a first conductor tip 110 and increasing a temperature of a second conductor tip 110 may facilitate thermal spread to healthy tissue. In one or more embodiments, increasing a temperature of a first conductor tip 110 and increasing a temperature of a second conductor tip 110 may be configured to cause a tissue to stick to the first conductor tip 110 and the second conductor tip 110. Illustratively, decreasing a temperature of a first conductor tip 110 and decreasing a temperature of a second conductor tip 110 may be configured to prevent a tissue from sticking to the first conductor tip 110 and the second conductor tip 110. In one or more embodiments, circulating a coolant through an internal conduit 160 of a first forceps arm 100 and circulating a coolant through an internal conduit 160 of a second forceps arm 110 may be configured to decrease a temperature of a first conductor tip 110 and decrease a temperature of a second conductor tip 110, e.g., circulating a coolant through an internal conduit 160 of a first forceps arm 100 and circulating a coolant through an internal conduit 160 of a second forceps arm 100 of may be configured to prevent a tissue from sticking to a first conductor tip 110 and a second conductor tip 110. Illustratively, circulating a coolant through a first conductor tip 110 and circulating a coolant through a second conductor tip 110 may be configured to decrease a temperature of the first conductor tip 110 and decrease a temperature of the second conductor tip 110, e.g., circulating a coolant through a first conductor tip 110 and circulating a coolant through a second conductor tip 110 may be configured to prevent a tissue from sticking to the first conductor tip 110 and the second conductor tip 110. In one or more embodiments, circulating a coolant into a first conductor tip 110 and out from the first conductor tip 110 and circulating a coolant into a second conductor tip 110 and out from the second conductor tip 110 may be configured to decrease a temperature of the first conductor tip 110 and decrease a temperature of the second conductor tip 110, e.g., circulating a coolant into a first conductor tip 110 and out from the first conductor tip 110 and circulating a coolant into a second conductor tip 110 and out from the second conductor tip 110 may be configured to prevent a tissue from sticking to the first conductor tip 110 and the second conductor tip 110. Illustratively, circulating a first coolant into a first conductor tip 110 and out from the first conductor tip 110 circulating a second coolant into a second conductor tip 110 and out from the second conductor tip 110 and may be configured to increase a temperature of the first coolant and increase a temperature of the second coolant and decrease a temperature of the first conductor tip 110 and decrease a temperature of the second conductor tip 110. In one or more embodiments, decreasing a temperature of a first conductor tip 110 and decreasing a temperature of a second conductor tip 110 may be configured to prevent thermal spread to a non-target tissue, e.g., decreasing a temperature of a first conductor tip 110 and decreasing a temperature of a second conductor tip 110 may be configured to prevent thermal spread to healthy tissue.

FIG. 4C illustrates conductor tips in a fully closed orientation 420. Illustratively, an application of a force to a lateral portion of forceps arms 100 may be configured to gradually close conductor tips 110 from conductor tips in a partially closed orientation 410 to conductor tips in a fully closed orientation 420. In one or more embodiments, first forceps arm conductor tip 110 and second forceps arm conductor tip 110 may have a contact area in a range of 0.01 to 0.015 square inches when conductor tips 110 comprise conductor tips in a fully closed orientation 420, e.g., first forceps arm conductor tip 110 and second forceps arm conductor tip 110 may have a contact area of 0.0125 square inches when conductor tips 110 comprise conductor tips in a fully closed orientation 420. Illustratively, first forceps arm conductor tip 110 and second forceps arm conductor tip 110 may have a contact area less than 0.01 square inches or greater than 0.015 square inches when conductor tips 110 comprise conductor tips in a fully closed orientation 420. Illustratively, an application of a force having a magnitude in a range of 1.5 to 3.3 pounds to a lateral portion of forceps arms 100 may be configured to gradually close conductor tips 110 from conductor tips in a partially closed orientation 410 to conductor tips in a fully closed orientation 420, e.g., an application of a force having a magnitude of 2.5 pounds to a lateral portion of forceps arms may be configured to gradually close conductor tips 110 from conductor tips in a partially closed orientation 410 to conductor tips in a fully closed orientation 420. In one or more embodiments, an application of a force having a magnitude less than 1.5 pounds or greater than 3.3 pounds to a lateral portion of forceps arms 100 may be configured to gradually close conductor tips 110 from conductor tips in a partially closed orientation 410 to conductor tips in a fully closed orientation 420.

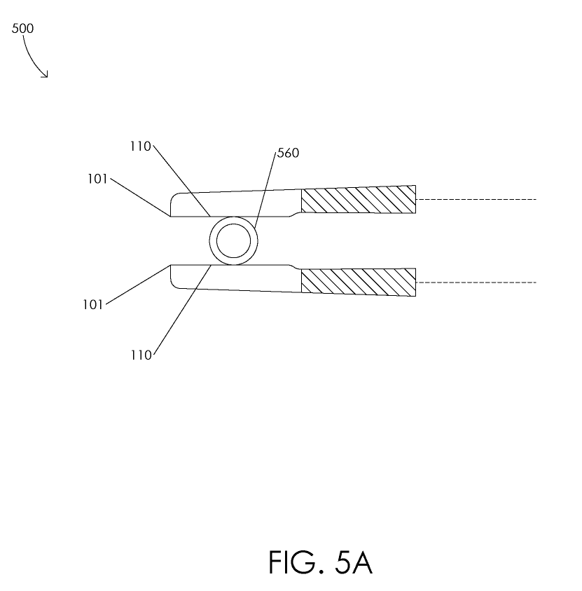

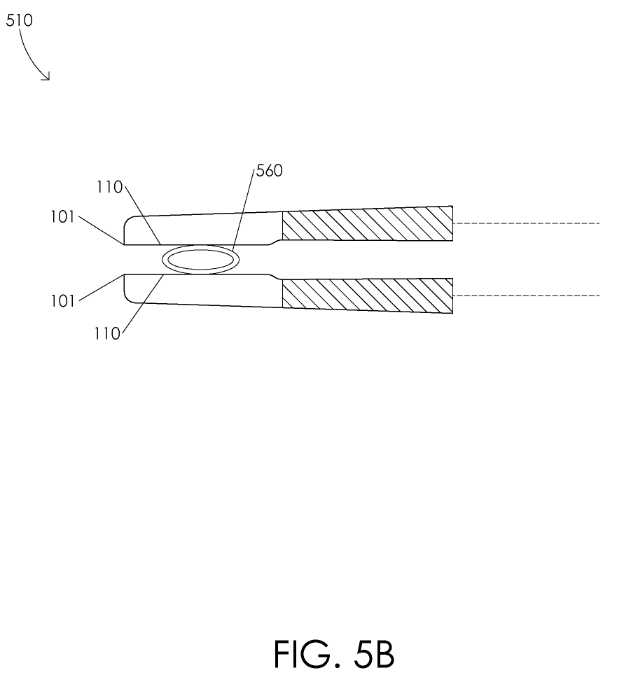

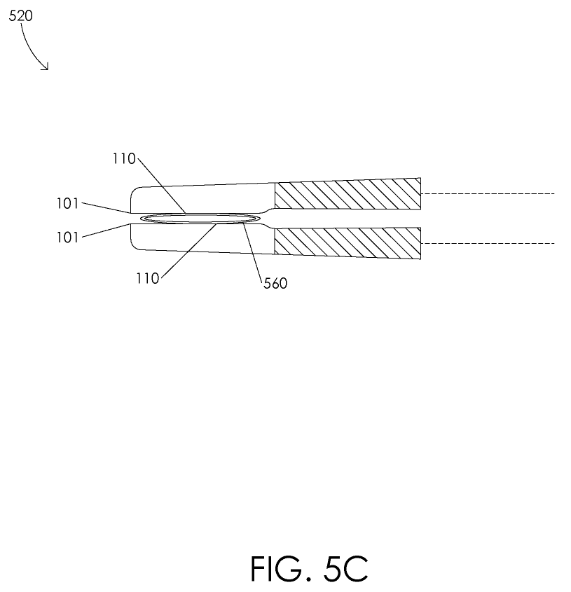

FIGS. 5A, 5B, and 5C are schematic diagrams illustrating a uniform compression of a vessel 560. In one or more embodiments, vessel 560 may comprise a blood vessel of an arteriovenous malformation. FIG. 5A illustrates an uncompressed vessel 500. Illustratively, vessel 560 may comprise an uncompressed vessel 500, e.g., when vessel 560 has a natural geometry. In one or more embodiments, vessel 560 may comprise an uncompressed vessel, e.g., when conductor tips 110 comprise conductor tips in a partially closed orientation 410. Illustratively, a surgeon may dispose vessel 560 between a first conductor tip 110 and a second conductor tip 110, e.g., when conductor tips 110 comprise conductor tips in an open orientation 400. In one or more embodiments, an application of a force to a lateral portion of forceps arms 100 may be configured to gradually close conductor tips 110 from conductor tips in an open orientation 400 to conductor tips in a partially closed orientation 410. Illustratively, vessel 560 may electrically connect a first conductor tip 110 and a second conductor tip 110, e.g., when vessel 560 comprises an uncompressed vessel 500. In one or more embodiments, a surgeon may identify an orientation of conductor tips 110 wherein conductor tips 110 initially contact vessel 560. Illustratively, a geometry of forceps arms 100 may be configured to allow a surgeon to visually identify an orientation of conductor tips 110 wherein conductor tips 110 initially contact vessel 560. In one or more embodiments, a mass of forceps arms 100 may be configured to allow a surgeon to tactilely identify an orientation of conductor tips 110 wherein conductor tips 110 initially contact vessel 560. Illustratively, a geometry of forceps arms 100 and a mass of forceps arms 100 may be configured to allow a surgeon to both visually and tactilely identify an orientation of conductor tips 110 wherein conductor tips 110 initially contact vessel 560.

FIG. 5B illustrates a partially compressed vessel 510. Illustratively, an application of a force to a lateral portion of forceps arms 100 may be configured to uniformly compress vessel 560 from an uncompressed vessel 500 to a partially compressed vessel 510. In one or more embodiments, an application of a force to a lateral portion of forceps arms 100 may be configured to uniformly increase a contact area between vessel 560 and forceps arm conductor tips 110. Illustratively, vessel 560 may electrically connect first forceps arm conductor tip 110 and second forceps arm conductor tip 110, e.g., when vessel 560 comprises a partially compressed vessel 510. In one or more embodiments, an application of a force to a lateral portion of forceps arms 100 may be configured to compress vessel 560 wherein vessel 560 maintains a symmetrical geometry with respect to a medial axis of vessel 560. Illustratively, vessel 560 may have a symmetrical geometry with respect to a medial axis of vessel 560 when vessel 560 comprises a partially compressed vessel 510. In one or more embodiments, conductor tips 110 may be configured to compress vessel 560 wherein no portion of vessel 560 is compressed substantially more than another portion of vessel 560, e.g., conductor tips 110 may be configured to evenly compress vessel 560 without pinching a first portion of vessel 560 or bulging a second portion of vessel 560. Illustratively, vessel 560 may be evenly compressed when vessel 560 comprises a partially compressed vessel 510.

FIG. 5C illustrates a fully compressed vessel 520. Illustratively, an application of a force to a lateral portion of forceps arms 100 may be configured to uniformly compress vessel 560 from a partially compressed vessel 510 to a fully compressed vessel 520. In one or more embodiments, an application of a force to a lateral portion of forceps arms 100 may be configured to uniformly increase a contact area between vessel 560 and forceps arm conductor tips 110. Illustratively, vessel 560 may electrically connect first forceps arm conductor tip 110 and second forceps arm conductor tip 110, e.g., when vessel 560 comprises a fully compressed vessel 520. In one or more embodiments, a surgeon may uniformly cauterize vessel 560, e.g., when vessel 560 comprises a fully compressed vessel 520. Illustratively, a surgeon may uniformly achieve hemostasis of vessel 560, e.g., when vessel 560 comprises a fully compressed vessel 520. In one or more embodiments, an application of a force to a lateral portion of forceps arms 100 may be configured to compress vessel 560 wherein vessel 560 maintains a symmetrical geometry with respect to a medial axis of vessel 560. Illustratively, vessel 560 may have a symmetrical geometry with respect to a medial axis of vessel 560 when vessel 560 comprises a fully compressed vessel 520. In one or more embodiments, conductor tips 110 may be configured to compress vessel 560 wherein no portion of vessel 560 is compressed substantially more than another portion of vessel 560, e.g., conductor tips 110 may be configured to evenly compress vessel 560 without pinching a first portion of vessel 560 or bulging a second portion of vessel 560. Illustratively, vessel 560 may be evenly compressed when vessel 560 comprises a fully compressed vessel 520.

The foregoing description has been directed to particular embodiments of this invention. It will be apparent; however, that other variations and modifications may be made to the described embodiments, with the attainment of some or all of their advantages. Specifically, it should be noted that the principles of the present invention may be implemented in any system. Furthermore, while this description has been written in terms of a surgical instrument, the teachings of the present invention are equally suitable to any systems where the functionality may be employed. Therefore, it is the object of the appended claims to cover all such variations and modifications as come within the true spirit and scope of the invention.

What is claimed is:

1. An instrument comprising:
a first forceps arm having a first forceps arm distal end and a first forceps arm proximal end;
a first forceps arm grip of the first forceps arm having a first forceps arm grip distal end and a first forceps arm grip proximal end wherein the first forceps arm grip distal end is disposed between the first forceps arm distal end and the first forceps arm proximal end and wherein the first forceps arm grip proximal end is disposed between the first forceps arm distal end and the first forceps arm proximal end;
a first conductor tip of the first forceps arm having a first conductor tip distal end and a first conductor tip proximal end;
a first input conductor housing of the first forceps arm;
a first coating of an electrical insulator material over at least a portion of the first forceps arm;
a first internal conduit of the first forceps arm having a first superior opening, a first inferior opening, and a first internal conduit distal end wherein the first internal conduit distal end is disposed within a portion of the first conductor tip;
a second forceps arm having a second forceps arm distal end and a second forceps arm proximal end, the second forceps arm disposed opposite the first forceps arm;
a second forceps arm grip of the second forceps arm having a second forceps arm grip distal end and a second forceps arm grip proximal end, the second forceps arm grip disposed opposite the first forceps arm grip wherein the second forceps arm grip distal end is disposed between the second forceps arm distal and the second forceps arm proximal end and wherein the second forceps arm grip proximal end is disposed between the second forceps arm distal end and the second forceps arm proximal end;
a second conductor tip of the second forceps arm having a second conductor tip distal end and a second conductor tip proximal end;
a second input conductor housing of the second forceps arm;
a second coating of the electrical insulator material over at least a portion of the second forceps arm;
a second internal conduit of the second forceps arm having a second superior opening, a second inferior opening, and a second internal conduit distal end wherein the second internal conduit distal end is disposed within a portion of the second conductor tip; and
a coolant multiplexer having a coolant transfer tube housing, the coolant multiplexer configured to electrically isolate the first input conductor housing of the first forceps arm and the second input conductor housing of the second forceps arm wherein the first forceps arm proximal end is disposed in the coolant multiplexer and the second forceps arm proximal end is disposed in the coolant multiplexer.

2. The instrument of claim 1 further comprising:
a superior coolant path of the first internal conduit having a superior coolant path proximal end; and
an inferior coolant path of the first internal conduit having an inferior coolant path proximal end.

3. The instrument of claim 2 further comprising:
a first superior coolant path interface having a first superior coolant path interface distal end and a first superior coolant path interface proximal end wherein the first superior coolant path interface distal end is disposed within the superior coolant path proximal end and wherein the first superior coolant path interface proximal end is disposed within the coolant multiplexer.

4. The instrument of claim 3 further comprising:
a first inferior coolant path interface having a first inferior coolant path interface distal end and a first inferior coolant path interface proximal end wherein the first inferior coolant path interface distal end is disposed within the inferior coolant path proximal end and wherein the first inferior coolant path interface proximal end is disposed within the coolant multiplexer.

5. The instrument of claim 4 further comprising:
a coolant transfer tube having a coolant transfer tube distal end and a coolant transfer tube proximal end wherein the coolant transfer tube distal end is disposed in the coolant transfer tube housing.

6. The instrument of claim 5 further comprising:
a coolant transfer machine interface configured to interface with a coolant transfer machine wherein a portion of the coolant transfer machine interface is disposed within the coolant transfer tube proximal end.

7. The instrument of claim 6 wherein the coolant transfer machine is configured to circulate a coolant into the first conductor tip and out from the first conductor tip.

8. The instrument of claim 7 wherein circulating the coolant into the first conductor tip and out from the first conductor tip is configured to decrease a temperature of the first conductor tip.

9. The instrument of claim 8 wherein decreasing the temperature of the first conductor tip is configured to prevent a tissue from sticking to the first conductor tip.

10. The instrument of claim 6 wherein the coolant transfer machine is configured to circulate a coolant through the first internal conduit.

11. The instrument of claim 10 wherein the coolant is configured to ingress the first internal conduit at the superior coolant path proximal end and wherein the coolant is configured to egress the first internal conduit at the inferior coolant path proximal end.

12. The instrument of claim 10 wherein the coolant is configured to ingress the first internal conduit at the inferior coolant path proximal end and wherein the coolant is configured to egress the first internal conduit at the superior coolant path proximal end.

13. The instrument of claim 10 wherein circulating the coolant through the first internal conduit is configured to decrease a temperature of the first conductor tip.

14. The instrument of claim 13 wherein decreasing the temperature of the first conductor tip is configured to prevent a tissue from sticking to the first conductor tip.

15. The instrument of claim 10 further comprising:
a bipolar cord; and
an electrosurgical generator adapter of the bipolar cord.

16. The instrument of claim 10 wherein the coolant is water.

17. The instrument of claim 10 wherein the coolant is ethylene glycol.

18. The instrument of claim 10 wherein the coolant is a nanofluid.

19. The instrument of claim 10 further comprising:
a thermally insulated portion of the coolant transfer tube.

20. The instrument of claim 10 wherein a coolant temperature of the coolant is lowered before circulating the coolant through the first internal conduit.
